(19) United States
(12) Patent Application Publication
(10) Pub. No.: US 2021/0147369 A1
(43) Pub. Date: May 20, 2021

(54) ACRYLATED UVA AND METHOD OF MAKING THE SAME

(71) Applicant: Madico, Inc., Pinellas Park, FL (US)

(72) Inventors: Yaroslav Klichko, Pinellas Park, FL (US); Philip Canale, Pinellas Park, FL (US); Marina Temchenko, Pinellas Park, FL (US)

(21) Appl. No.: 17/099,659

(22) Filed: Nov. 16, 2020

Related U.S. Application Data
(60) Provisional application No. 62/935,143, filed on Nov. 14, 2019.

(51) Int. Cl. C07D 251/24; C09D 175/16

(52) U.S. Cl. CPC C07D 251/24; C09D 175/16

(57) ABSTRACT

A UV absorber-comprising urethane acrylate of formula (I) and a HALS-comprising urethane acrylate of formula (I):

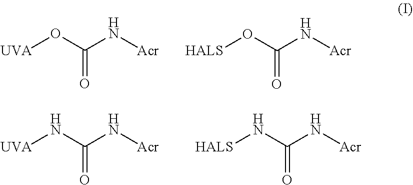

wherein, UVA is a UV absorber molecule that has a hydroxyl or an amine group reactive with isocyanate, HALS is a HALS molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate. The UV absorber-comprising urethane exhibits a UV absorption maximum between 300 and 360 nanometers.

### CROSS REFERENCE TO RELATED APPLICATION(S)

This application claims the benefit of U.S. Provisional Application No. 62/935,143, filed on Nov. 14, 2019. The entire disclosure of the above application is hereby incorporated herein by reference.

### FIELD

The present invention relates generally to an ultraviolet absorber (UV) and hindered amine light stabilizers(HALS), and more particularly to a UV absorber-comprising urethane acrylate and HALS-comprising urethane acrylate, a process of preparing the same, and to a use thereof. The UV absorber or HALS covalently bonded into the system by means of alkene functionalization.

### BACKGROUND OF THE INVENTION

Currently, transparent plastics or polymer articles, such as, for example, sheets, films or extruded moldings, must be protected especially by means of ultraviolet (UV) protection against exposure to aggressive solar radiation and by means of a scratch-resistant finish or coating to militate against exposure to mechanical disturbance or other types of physical and natural disturbances. Commonly, the upper or outermost layer or coating of the plastic articles, which receives the initial and most exposure to ultraviolet creating means and mechanical disturbance, is the protective layer. The protective layer is the layer that includes a substantial amount of UV absorbers and hindered amine light stabilizer (HALS). Often, the upper or outer layer is the only layer of the plastic articles considered a protective layer. Conventional UV absorbers and HALS, however, act as plasticizers in the protective layers and reduce the mechanical resistance of the layer. If not reacted into the backbone of the plastic polymer, additional problems exist such as blooming or hazing caused by in situ precipitation of the UV absorber or HALS into the solid state of the polymerized matrix.

Reacting UV absorbers and HALS into the polymer eliminates the migration of the compounds forming the UV absorbers and HALS to the surface of the polymer mitigating potential hazardous exposure to these chemicals.

HALS imparts UV protection to the coating. However, unlike UV absorbers, which absorb UV radiation, HALS eliminates the free radicals that are formed in the coating, thus providing protection to the coating or outermost layer as well. UV absorbers and HALS are typically employed together.

Typical UV absorber classes are, for example, phenyl-substituted triazines, hydroxyphenyl-benzotriazole. These substances exhibit an outstanding absorption effect of UV radiation as well as a very high intrinsic UV stability.

The influence of the UV absorbers and HALS, in accordance with Beer-Lambert Law, on the mechanical and chemical resistance of the coating is greater the higher the concentration thereof in the coating. However, certain concentrations of the UV absorbers are required in order to absorb UV light effectively and protect the plastic or polymer substrate from the UV light reliably. According to Beer-Lambert's Law, concentration of the UV absorbers should be higher, the thinner the coating in order to absorb UV light as desired. For modern substrate coatings, the outer layer thickness can be between about 1 and 50 μm and including up to 10 wt. % of the UV absorber in order to exhibit the necessary absorbing power. However, the mechanical and chemical resistance of such coatings could be adversely affected from such a concentrations of the UV absorbers.

A possible solution to the problem might be for the UV absorber and HALS not to be present as a passive additive but to play an active part chemically in the curing process and to be incorporated into the polymer framework of the coating.

Therefore, it is desirable to actively incorporate UV absorbers and HALS chemically into a polymer framework by chemically modifying the UV absorbers and HALS to add acrylate functionality. As a result, UV protection, chemical resistance, and mechanical resistance of the polymer and coating together is maximized.

### SUMMARY OF THE INVENTION

In accordance and attuned with the present invention, a structure, system, and process of actively incorporating the UV absorbers and the HALS chemically into the polymer framework by chemically modifying the UV absorbers and HALS to add acrylate functionality has been discovered.

According to a first embodiment, a UV absorber-comprising urethane acrylate of formula (I) is disclosed. Formula (I) is as follows:

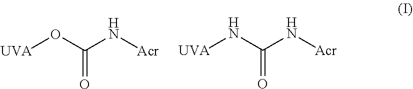

wherein UVA is a UV absorber molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate.

According to a second embodiment of the disclosure a HALS-comprising urethane acrylate of formula (I) id disclosed. Formula (I) is as follows:

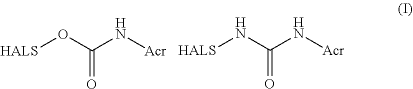

wherein HALS is a HALS molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate.

According to a third embodiment of the disclosure, a process for preparation of a UV absorber-comprising urethane acrylate, comprises the steps of: providing a UV-absorber compound containing hydroxyl group capable of reacting with isocyanate and reacting the UV-absorber compound containing hydroxyl with a dual-functional monomer containing acrylate and isocyanate groups according to the following reaction scheme:

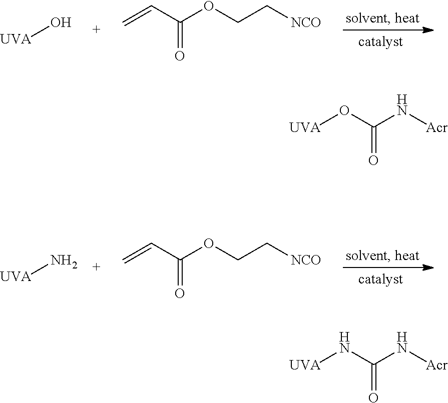

wherein, UVA is the UV-absorber compound, and Acr is an acrylate or methacrylate group, and wherein, the reaction is carried out in a solvent and a catalyst is employed to accelerate the reaction.

According to a fourth embodiment of the disclosure, a process for preparation of a HALS-comprising urethane acrylate, comprises the steps of: providing a HALS compound containing hydroxyl group capable of reacting with isocyanate and reacting the HALS containing hydroxyl with a dual-functional monomer containing acrylate and isocyanate groups according to the following reaction scheme:

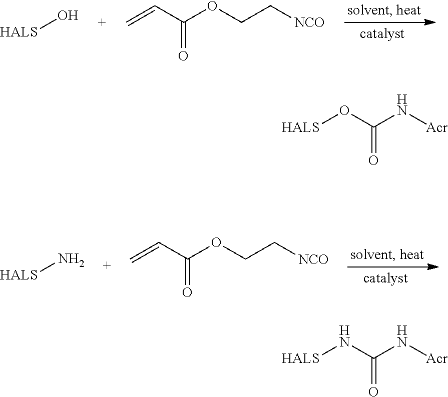

wherein, HALS is the HALS compound, and Acr is an acrylate or methacrylate group, and wherein, the reaction is carried out in a solvent and a catalyst is employed to accelerate the reaction.

According to a fifth embodiment of the disclosure, a coating composition comprising a UV absorber-comprising urethane acrylate according to formula (I) or a HALS-comprising urethane acrylate according to formula (II) id disclosed. The formula (I) and the formula (II) are as follows:

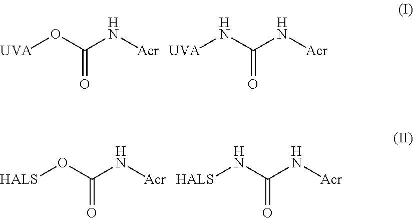

Wherein UVA is a UV absorber molecule that has a hydroxyl or an amine group reactive with isocyanate, HALS is a HALS molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate.

### DESCRIPTION OF THE PREFERRED EMBODIMENT

The following detailed description and appended drawings describe and illustrate various exemplary embodiments of the invention. The description and drawings serve to enable one skilled in the art to make, and use the invention, and are not intended to limit the scope of the invention in any manner. With respect to the methods disclosed, the steps presented are exemplary in nature, and thus, the order of the steps is not necessary or critical.

The present invention relates to ultraviolet (UV) absorbers including urethane acrylate and hindered amine light stabilizers (HALS) including urethane acrylate, to a process for the preparation of the UV absorbers including urethane acrylate and the HALS including urethane acrylate, and the use of UV absorbers including urethane acrylate and HALS including urethane acrylate. As used herein, the UV absorber-comprising urethane acrylate has a Formula (1):

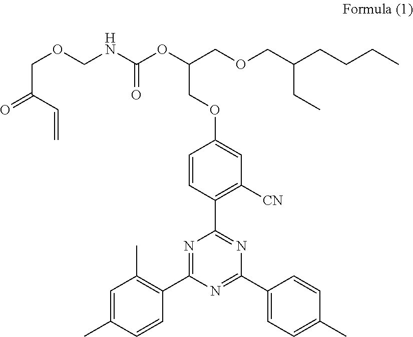 [SMILES: C=CC(=O)COCNC(=O)OC(COCC(CC)CCCC)COc1ccc(-c2nc(-c3ccc(C)cc3)nc(-c3ccc(C)cc3C)n2)c(C#N)c1]

As used herein, the HALS-comprising urethane acrylate has a Formula (2).

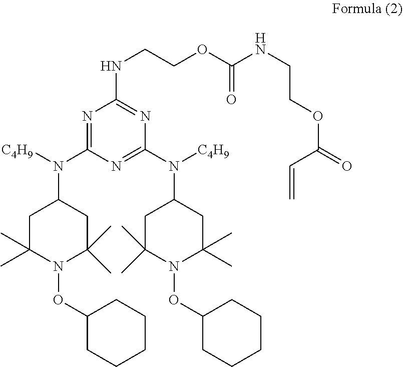

As used herein, for exemplary purposes, the UV absorber-comprising urethane acrylate and HALS-comprising urethane acrylate may be employed as coatings on or comprised within polymers for UV protection. Although it is understood, the UV absorber-comprising urethane acrylate and the HALS-comprising urethane acrylate may facilitate the UV protection of other applications, materials, chemicals, compounds, or the like.

The object of the present disclosure has been achieved by a UV absorber-comprising urethane acrylate of a general Formula (3).

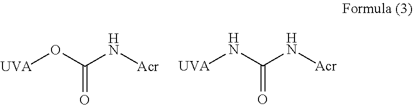

wherein, UVA is a UV absorber molecule that has a hydroxyl or an amine group reactive with isocyanate. Acr is an acrylate, methacrylate or ethacrylate group attached through a linker of commercially available linkers such as 2-isocyanatoethyl acrylate, 2-isocyanatoethyl methacrylate, 2-isocyanatoethyl ethacrylate, or 1,1-Bis(acryloxymethyl)ethyl isocyanate, for example.
The compounds of the formula (3) according to the invention preferably exhibit a UV absorption maximum between 300 and 360 nm.

The HALS-comprising urethane acrylate has a general Formula (4):

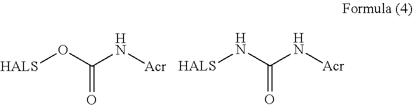

### Wherein,

HALS is a hindered amine light stabilizer molecule that has a hydroxyl or an amine group reactive capable of reacting with the isocyanate moiety. Acr is an acrylate or methacrylate group attached through a linker of commercially available linkers such as 2-isocyanatoethyl acrylate, 2-isocyanatoethyl methacrylate, 2-isocyanatoethyl ethacrylate, or 1,1-Bis(acryloxymethyl)ethyl isocyanate. Examples (example 1 and example 2) of structures are shown below in FIG. 1.

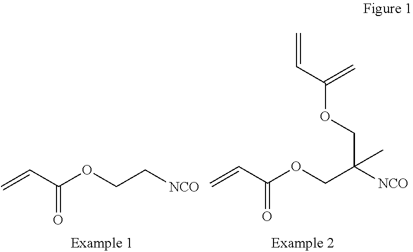

The present invention further provides a process for the preparation of the UV absorber-comprising urethane acrylate, and a HALS-comprising urethane acrylate as follows:
i. a UV absorber compound containing hydroxyl or amine group available for reaction with isocyanate, is reacted with a dual-functional monomer containing acrylate and isocyanate groups, an example of which is shown in Scheme 1.
ii. a HALS compound containing hydroxyl or amine group available for reaction with isocyanate, is reacted with a dual-functional monomer containing acrylate and isocyanate groups, an example of which is shown in Scheme 2.

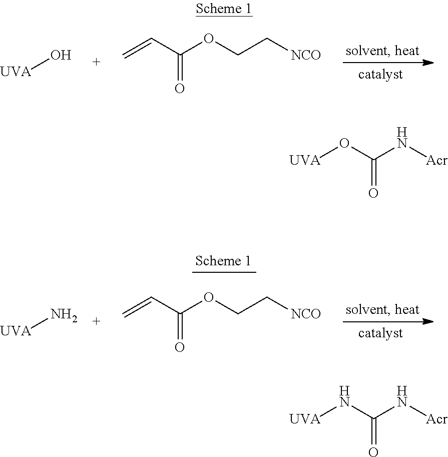

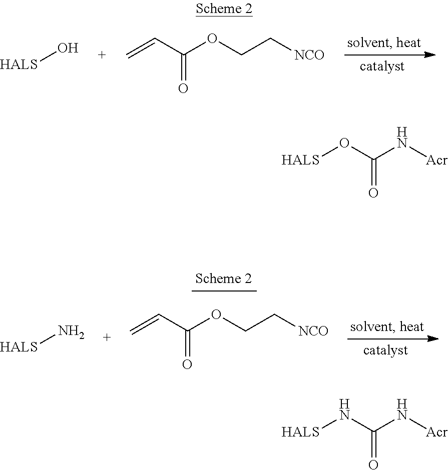

The reaction is carried out in a suitable solvent. A suitable catalyst, for those skilled in the art of urethane chemistry, may be used to accelerate the reaction.

A dual-functional monomer containing acrylate and isocyanate groups includes but not limited to 2-isocyanatoethyl acrylate, 2-isocyanatoethyl methacrylate 2-isocyanatoethyl ethacrylate, and/or 1,1-Bis(acryloxymethyl)ethyl isocyanate. Some examples of which are shown in FIG. 2.

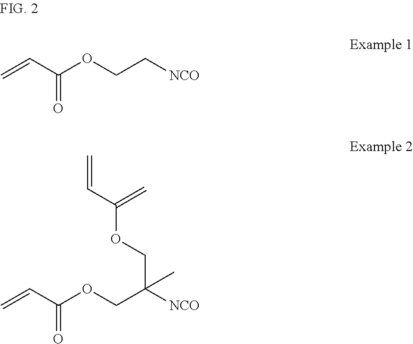

Also, an isocyanate functional urethane acrylate polymer can be used in a reaction. Examples of isocyanate functional urethane polymers include, for example, Ebecryl 4397, Ebecryl 4396, Ebecryl 4250, Ebecryl 4150, and Sartomer CN9302.

The limitation of the solvent used for the reactions is that the solvent does not react with isocyanate groups under typical process conditions. Typical examples of the isocyanate groups that do not react are known to the person skilled in the art. They are aromatic hydrocarbons (toluene), cyclic ethers (THF, dioxane), ketones (acetone, MEK, cyclopentanone) or esters (ethyl and butyl acetate).

At the end of the process, if the resin of Formula (1) and (2) no longer possesses any free NCO groups, solvents are not subject to the limitations mentioned hereinabove above. Accordingly, these solvents include those mentioned above plus all OH-containing solvents known to the person skilled in the art, such as, for example, ethanol, isopropanol, butanol, 1-methoxy-2-propanol, and mixtures thereof.

Upon completion of the reaction, the reaction solution can be used directly in coating formulations without any purification or isolation steps as the amount of impurities or byproducts is very low or minimized.

Certain structures, for example, as shown in EP 2 951 162 Bland U.S. Pat. No. 9,604,943 B2, the disclosures of which are herein incorporated by reference in their entirety, use a hydroxyl containing acrylate and a difunctional isocyanate to produce similar structures as proposed herein. However, many side products are yielded in these reactions unless prior purification steps are used between reaction steps.

However, according to the present disclosure, an alternate method using alternate materials will produce materials of superior purity without purification steps using a one-step simple synthesis.

The present disclosure relates further to a coating composition comprising a UV absorber-comprising urethane acrylate and HALS-comprising urethane acrylate according to the invention and/or a UV absorber-comprising urethane acrylate and HALS-comprising urethane acrylate obtainable by a process according to the instant disclosure.

According to one embodiment, the coating composition can include the following:
i. A UV absorber-comprising urethane acrylate according to the present invention and/or a UV absorber-comprising urethane acrylate obtainable by a process according to the present invention in an amount of from 0 to 30 parts by weight, preferably from 1 to 12 wt. %, based on the total amount in the final coating composition (processed, meaning dried and polymerized).
ii. HALS-comprising urethane acrylate according to the present invention and/or a HALS-comprising urethane acrylate obtainable by a process according to the present invention in an amount of from 0 to 25 parts by weight (preferably from 0.5 to 5 wt. %, based on the total amount of the coating composition).
iii. From 0 to 50 parts by weight of at least one C₂-C₁₂-diol diacrylate or C₂-C₁₂-diol dimethacrylate, wherein C₂-C₂ represents a linear alkylene radical which can be optionally substituted by a methyl group or can be interrupted by one or more oxygen atom(s) and optionally substituted by one or more methyl group(s).
iv. From 0 to 95 parts by weight of at least one alkoxylated, preferably ethoxylated, mono-, di-, tri-, tetra-, penta- or hexa-acrylate or alkoxylated, preferably ethoxylated, mono-, di-, tri-, tetra-, penta- or hexa-methacrylate.
v. From 0 to 95 parts by weight of at least one monomer selected from the group comprising pentaerythritol triacrylate, pentaerythritol tetraacrylate, dipentaerythritol tetraacrylate, dipentaerythritol pentaacrylate, dipentaerythritol hexaacrylate, pentaerythritol trimethacrylate, pentaerythritol tetramethacrylate, dipentaerythritol tetramethacrylate, dipentaerythritol pentamethacrylate, dipentaerythritol hexamethacrylate and possible reaction products thereof with aliphatic or aromatic diisocyanates preferably aliphatic, producing urethane acrylates.
vi. From 0 to 95 parts by weight of at least one further mono-, di- or tri-acrylate or mono-, di- or tri-methacrylate, wherein the sum of the parts by weight of components i. to v. is ≤100 parts by weight, and the coating composition additionally comprises at least
vii. From 0.1 to 10 parts by weight of at least one photoinitiator or thermal free radical initiator.

Component iii) is preferably at least one C4-C12-diol diacrylate or C4-C12-diol dimethacrylate, particularly preferably at least one C4-C8-diol diacrylate or C4-C8-diol dimethacrylate. The C2-C12, preferably C4-C12, particularly preferably C4-C8, units are preferably linear alkylene radicals which can be optionally substituted by a methyl group or can be interrupted by one or more oxygen atom(s) and optionally substituted by one or more methyl group(s). They are preferably linear alkene radicals which can optionally be interrupted by one or more oxygen atom(s).

Suitable diol diacrylates or diol dimethacrylates are most particularly preferably those of a general formula (5).

H2C═C(R2)-C(O)—O—(CH₂)ₙ—O—C(O)—C(R2)=CH₂ Formula (5):

wherein, R2 represents 1-1 or CH₃, n represents an integer from 2 to 12, preferably from 4 to 12, and particularly preferably from 4 to 8.

Further suitable diol diacrylates or diol dimethacrylates are most particularly preferably those of a general formula (6).

H₂C═C(R3)-C(O)—O—(CHR4-CH₂—O)ₖ—C(O)—C(R3)=CH₂ Formula (6):

wherein, R3 represents H or CH3, preferably H, R4 represents H or CH3, and k represents an integer from 2 to 5, preferably from 2 to 4.

Suitable C4-C8-diol diacrylates or -diol dimethacrylates are, for example, diethylene glycol diacrylate, diethylene glycol dimethacrylate, triethylene glycol diacrylate, triethylene glycol dimethacrylate, tetraethylene glycol diacrylate, tetraethylene glycol dimethacrylate, dipropylene glycol diacrylate, dipropylene glycol dimethacrylate, tripropylene glycol diacrylate, tripropylene glycol dimethacrylate, 1,4-butanediol diacrylate, 1,4-butanediol dimethacrylate, 1,5-pentanediol diacrylate, 1,5-pentanediol dimethacrylate, 1,6-hexanediol diacrylate, 1,6-hexanediol dimethacrylate, 3-methyl-1,5-pentanediol diacrylate, 3-methyl-1,5-pentanediol dimethacrylate, 1,7-heptanediol diacrylate, 1,7-heptanediol dimethacrylate, 1,8-octanediol diacrylate and/or 1,8-octanediol dimethacrylate. C4-C12-Diol diacrylates or -diol dimethacrylates which are additionally suitable are, for example, 1,9-nonanediol diacrylate, 1,9-nonanediol dimethacrylate, 2-methyl-1,8-octanediol diacrylate, 2-methyl-1,8-octanediol dimethacrylate, 1,10-decanediol diacrylate, 1,10-decanediol dimethacrylate, 1,11-undecanediol diacrylate, 1,11-undecanediol dimethacrylate, 1,12-dodecanediol diacrylate and/or 1,12-dodecanediol dimethacrylate. C2-C12-Diol diacrylates or -diol dimethacrylates which are additionally suitable are, for example, ethylene glycol diacrylate, ethylene glycol dimethacrylate, 1,3-propanediol diacrylate and/or 1,3-propanediol dimethacrylate. Most particular preference is given to the aliphatic diacrylates in each case. Particular preference is given to 1,6-hexanediol diacrylate and/or 1,6-hexanediol dimethacrylate, diethylene glycol diacrylate, diethylene glycol dimethacrylate, particularly preferably 1,6-hexanediol diacrylate, diethylene glycol diacrylate.

The coating composition comprises alkoxylated mono-, di-, tri-, tetra, penta, or hexa-acrylates or -methacrylates of which component iv) is composed.

Typical of the lower functional (mono) acrylate structural chemical formula are the alkoxylated monoacrylates or monomethacrylates for component (iv) can be alkoxylated optionally substituted aliphatic, cycloaliphatic, aromatic, mixed aromatic-aliphatic monoacrylates or monomethacrylates. There are suitable both alkoxylated linear and branched aliphatic monoacrylates or monomethacrylates, in which the alkyl chain can additionally be interrupted by one or more heteroatoms, such as, for example, oxygen atoms. In the case of cycloaliphatic or aromatic monoacrylates or monomethacrylates, heterocyclic or heteroaromatic monoacrylates or monomethacrylates are also suitable.

Examples of such alkoxylated monoacrylates or monomethacrylates are alkoxylated, preferably ethoxylated, methyl acrylate, ethyl acrylate, n-butyl acrylate, isobutyl acrylate, tert-butyl acrylate, 2-ethylhexyl acrylate, isodecyl acrylate, n-lauryl acrylate, C12-C15-alkyl acrylates, n-stearyl acrylate, n-butoxyethyl acrylate, butoxy diethylene glycol acrylate, methoxy triethylene glycol acrylate, cyclohexyl acrylate, tetrahydrofurfuryl acrylate, benzyl acrylate, 2-phenoxyethyl acrylate, isobornyl acrylate, 2-hydroxyethyl acrylate, 2-hydroxypropyl acrylate, 2-hydroxyethyl acrylate, 2-hydroxybutyl acrylate, 2-hydroxybutyl acrylate and the corresponding alkoxylated, preferably ethoxylated, methacrylate.

Examples of such alkoxylated diacrylates or dimethacrylates are alkoxylated, preferably ethoxylated, methanediol diacrylate, methanediol dimethacrylate, glycerol diacrylate, glycerol dimethacrylate, neopentyl glycol di acrylate, neopentyl glycol dimethacrylate, 2-butyl-2-ethyl-1,3-propanediol diacrylate, 2-butyl-2-ethyl-1,3-propanediol dimethacrylate, trimethylolpropane di acrylate or trimethylolpropane dimethacrylate.

Examples of alkoxylated triacrylates or trimethacrylates for component (iv) are alkoxylated, preferably ethoxylated, pentaerythritol triacrylate, pentaerythritol trimethacrylate, glycerol triacrylate, glycerol trimethacrylate, 1,2,4-butanetriol triacrylate, 1,2,4-butanetriol trimethacrylate, trimethylolpropane triacrylate, trimethylolpropane trimethacrylate, tricyclodecanedimethanol diacrylate, tricyclodecanedimethanol dimethacrylate, ditrimethylolpropane tetraacrylate or ditrimethylolpropane tetramethacrylate.

Examples of alkoxylated tetra-, penta- or hexa-acrylates are alkoxylated, preferably ethoxylated, pentaerythritol tetraacrylate, dipentaerythritol tetraacrylate, dipentaerythritol pentaacrylate, dipentaerythritol hexaacrylate, pentaerythritol tetramethacrylate, dipentaerythritol tetramethacrylate, dipentaerythritol pentamethacrylate or dipentaerythritol hexamethacrylate.

In the alkoxylated diacrylates or dimethacrylates, triacrylates or trimethacrylates, tetraacrylates or tetramethacrylates, pentaacrylates or pentamethacrylates and/or alkoxylated hexaacrylates or hexamethacrylates of component iv), all the acrylate groups or methacrylate groups or only some of the acrylate groups or methacrylate groups in the monomer in question can be bonded via alkylene oxide groups to the corresponding radical. Arbitrary mixtures of such wholly or partially alkoxylated di-, tri-, tetra-, penta- or hexa-acrylates and -methacrylates can also be used. It is also possible for the acrylate or methacrylate group(s) to be bonded to the aliphatic, cycloaliphatic or aromatic radical of the monomer via a plurality of successive alkylene oxide groups, preferably ethylene oxide groups. The mean number of alkylene oxide or ethylene oxide groups in the monomer is given by the degree of alkoxylation or degree of ethoxylation. The degree of alkoxylation or degree of ethoxylation can preferably be from 2 to 25, particular preference being given to degrees of alkoxylation or degrees of ethoxylation of from 2 to 15, most particularly preferably from 3 to 9.

Component (iv) preferably comprises alkoxylated, preferably ethoxylated, di- and/or tri-acrylates. Component (iv) particularly preferably comprises at least one alkoxylated, preferably ethoxylated, di- or tri-acrylate or at least one alkoxylated, preferably ethoxylated, di- or tri-methacrylate, most particularly preferably an ethoxylated di- or tri-acrylate. In preferred embodiments of the invention, component (iv) comprises at least one ethoxylated triacrylate or trimethacrylate, preferably ethoxylated triacrylate. Particularly preferably, component (iv) comprises alkoxylated trimethylolpropane triacrylate and/or trimethylolpropane trimethacrylate. In preferred embodiments, component (iv) comprises ethoxylated trimethylolpropane triacrylate and/or trimethylolpropane trimethacrylate, preferably ethoxylated trimethylolpropane triacrylate.

The coating composition comprises preferably from 0 to 95 parts by weight, particularly preferably from 20 to 80 parts by weight, of component v). In particularly preferred embodiments, component iv) is present in the coating composition. The mentioned parts by weight are the sum of the parts by weight of all the monomers from the mentioned group of which component v) is composed.

Component v) is monomers selected from the group comprising pentaerythritol triacrylate, pentaerythritol tetraacrylate, dipentaerythritol tetraacrylate, dipentaerythritol pentaacrylate, pentaerythritol trimethacrylate, pentaerythritol tetramethacrylate, dipentaerythritol tetramethacrylate, dipentaerythritol pentamethacrylate and reaction products thereof with aliphatic or aromatic diisocyanates, as well as comprising dipentaerythritol hexaacrylate and dipentaerythritol hexamethacrylate. Component v) is preferably mixtures comprising two or more of the monomers mentioned above.

In embodiments that are most preferred, component v) comprises pentaerythritol triacrylate, pentaerythritol tetraacrylate, dipentaerythritol tetraacrylate, dipentaerythritol pentaacrylate and/or dipentaerythritol hexaacrylate.

The coating composition comprises preferably from 0 to 95 parts by weight, particularly preferably from 0 to 60 parts by weight, of component vi). The mentioned parts by weight are the sum of the parts by weight of all the mono-, di- or tri-acrylates or -methacrylates of which component vi) is composed.

The monoacrylates or monomethacrylates for component (v) can be optionally substituted aliphatic, cycloaliphatic, aromatic, mixed aromatic-aliphatic monoacrylates or monomethacrylates. There are suitable both linear and branched aliphatic monoacrylates or monomethacrylates, in which the alkyl chain can additionally be interrupted by one or more heteroatoms, such as, for example, oxygen atoms.

In the case of cycloaliphatic or aromatic monoacrylates or monomethacrylates, heterocyclic or heteroaromatic monoacrylates or monomethacrylates are also suitable. The monoacrylates or monomethacrylates which are suitable for component (v) are not alkoxylated.

Examples of such monoacrylates or monomethacrylates are methyl acrylate, ethyl acrylate, n-butyl acrylate, isobutyl acrylate, tert-butyl acrylate, 2-ethyl-hexyl acrylate, isodecyl acrylate, n-lauryl acrylate, C12-C15-alkyl acrylates, n-stearyl acrylate, n-butoxyethyl acrylate, butoxy diethylene glycol acrylate, methoxy triethylene glycol acrylate, cyclohexyl acrylate, tetrahydrofurfuryl acrylate, benzyl acrylate, 2-phenoxyethyl acrylate, isobornyl acrylate, 2-hydroxyethyl acrylate, 2-hydroxypropyl acrylate, 2-hydroxyethyl acrylate, 2-hydroxybutyl acrylate, 2-hydroxybutyl acrylate and the corresponding methacrylates.

The diacrylates or dimethacrylates for component (vi) can be, for example, those which are different from the diol diacrylates and dimethacrylates of component iii) and are not alkoxylated.

Examples of such diacrylates or dimethacrylates are methanediol diacrylate, methanediol dimethacrylate, glycerol diacrylate, glycerol dimethacrylate, neopentyl glycol diacrylate, neopentyl glycol dimethacrylate, 2-butyl-2-ethyl-1,3-propandiol diacrylate, 2-butyl-2-ethyl-1,3-propanediol dimethacrylate, trimethylolpropane diacrylate or trimethylolpropane dimethacrylate.

The triacrylates or trimethacrylates for component (vi) can be, for example, those which are different from the triacrylates and trimethacrylates of component v) and are not alkoxylated.

Examples of such triacrylates or trimethacrylates are glycerol triacrylate, glycerol trimethacrylate, 1,2,4-butanetriol triacrylate, 1,2,4-butanetriol trimethacrylate, trimethylolpropane triacrylate, trimethylolpropane trimethacrylate, tricyclodecanedimethanol diacrylate, tricyclodecanedimethanol dimethacrylate, ditrimethylolpropane tetraacrylate or ditrimethylolpropane tetramethacrylate.

Component (vi) preferably comprises at least one di- or tri-acrylate or at least one di- or tri-methacrylate. Component (v) particularly preferably comprises at least one triacrylate or trimethacrylate. Particularly preferably, component (vi) comprises trimethylolpropane triacrylate and/or trimethylolpropane trimethacrylate, preferably trimethylolpropane triacrylate.

Suitable photoinitiators (UV-driven initiators) preferably have a high photochemical reactivity and an absorption band in the near-UV range (>300 nm and particularly preferably >350 nm).

Suitable photoinitiators can be of any of the type I or type II photoinitiators available but are preferably those selected from the group of acylphosphine oxide derivatives, α-aminoalkylphenone derivatives, hydroxyalkylphenones, benzophenones, benzil ketals, methylbenzoyl formate and phenylacetophenones.

Examples of such photoinitiators are benzophenone, bis(2,4,6-trimethylbenzoyl)phenylphosphine oxide (Irgacure® 819 from BASF AG), 1-hydroxycyclohexyl phenyl ketone (Irgacure® 184 from BASF AG), 2-benzyl-2-(dimethylamino)-1-(4-morpholinophenyl)-1-butanone (Irgacure® 369 from BASF AG), 2-methyl-1-[4-(methylthio)phenyl]-2-morpholino-1-propanone (Irgacure® 907 from BASF AG), (1-hydroxycyclohexyl)phenylmethanone (Irgacure® 1800 from BASF AG), 2-hydroxy-2-methyl-1-phenyl-1-propanone (Irgacure® 1700 from BASF AG), bis(2,6-dimethylbenzoyl)(2,4,4-trimethylpentyl)phosphine oxide, bis(2,6-dimethoxybenzoyl)(2,4,4-trimethylpentyl)phosphine oxide, (2,4,6-trimethylbenzoyl)diphenylphosphine oxide (Lucirin® TPO Solid from BASF AG), 2,4,6-trimethylbenzoylethoxyphenylphosphine oxide (Lucirin® TPO-L from BASF AG), benzoylphosphonic acid bis(2,6-dimethylphenyl) ester (Lucirin® 8728 from BASF AG), and 2-hydroxy-2-methyl-1-phenyl-1-propanone (Darocur® 4265 from BASF AG).

Also suitable are mixtures of those photoinitiators with one another.

Typical thermal initiators are from the azide or peroxide family typical of which are dicumyl peroxide, benzoyl peroxide, and Azobisisobutyronitrile.

The coating composition can additionally comprise, in addition to the 100 parts by weight of components i) to vii), optionally one or more coating additives. Such coating additives can be selected, for example, from the group comprising stabilisers, flow agents, surface-active additives, pigments, dyes, inorganic nanoparticles, adhesion promoters, IR absorbers, preferably from the group comprising stabilisers, flow agents, surface-active additives such as slip agents which reduce the coefficient of friction, antifog agents which can reduce or illiminate fogging, or light minipulating agents of varying refractive index which may manipulate reflectance, and inorganic nanoparticles. The coating composition preferably comprises, in addition to the amount of photoinitiator or thermal initiator and in addition to the 100 parts by weight of components i) to vii), from 0 to 20 parts by weight, particularly preferably from 0 to 10 parts by weight, most particularly preferably from 0.1 to 10 parts by weight, of at least one further coating additive as component viii). Preferably, the total amount of all coating additives present in the coating composition is from 0 to 20 parts by weight, particularly preferably from 0 to 10 parts by weight, most particularly preferably from 0.1 to 10 parts by weight.

The coating composition can comprise inorganic nanoparticles for increasing the mechanical resistance, such as, for example, scratch resistance and/or pencil hardness.

Suitable nanoparticles are inorganic oxides, mixed oxides, hydroxides, sulfates, carbonates, carbides, borides and nitrides of elements of main groups II to IV and/or elements of subgroups I to VIII of the periodic system including the lanthanides. Preferred nanoparticles are silicon dioxide, aluminium oxide, cerium oxide, zirconium oxide, niobium oxide, zinc oxide or titanium oxide nanoparticles; silicon dioxide nanoparticles are particularly preferred.

The particles used preferably have mean particle sizes (measured by means of dynamic light scattering in dispersion determined as the Z-average) smaller than 200 nm, preferably from 5 to 100 nm, particularly preferably from 5 to 50 nm. Preferably at least 75%, particularly preferably at least 90%, most particularly preferably at least 95%, of all the nanoparticles used have the sizes defined above.

The coating composition can additionally comprise, in addition to the 100 parts by weight of components i) to vii), optionally one or more organic solvents or water. Such organic solvents can be selected, for example, from the group comprising aromatic solvents, such as, for example, xylene or toluene, ketones, such as, for example, acetone, 2-butanone, methyl isobutyl ketone, diacetone alcohol, alcohols, such as, for example, methanol, ethanol, isopropanol, butanol, 2-methoxy-propyl alcohol, ethers, such as, for example, 1,4-dioxane, ethylene glycol n-propyl ether, or esters, such as, for example, ethyl acetate, butyl acetate, 1-methoxy-2-propyl acetate, or mixtures comprising those solvents. The coating composition preferably comprises, in addition to the amount of photoinitiator and in addition to the 100 parts by weight of components i) to vii), from 0 to 300 parts by weight of at least one organic solvent as component ix).

The coating compositions can be prepared in a simple manner either by combining the individual components i) to vii) and, where appropriate, the optional components viii) and ix) in the absence of solvent(s) and mixing them together by stirring or, in the presence of solvent(s), by introducing them into the solvent or solvents, for example, and mixing them together by stirring. Optionally, purification by means of filtration, preferably by means of fine filtration, is then carried out.

The liquid form of the UV absorber and HALS (Formula 1 and 2) makes the admixture of components i) to vii) particularly simple, and the adapted structure of the absorber makes the resulting finished coating stable and transparent in all steps of the coating process and as a result particularly suitable for the production of the UV-protecting, weather-resistant, clear and hard surface of a plastics part or of a film.

The present invention further provides a method for coating a substrate, comprising the steps:
applying a coating composition according to the disclosure to a substrate,
curing the previously applied coating composition by irradiation with UV light.

Preferably, in the first step, the composition is applied to the surface of the substrate by any number of coating methods such as knife over roll, reverse gravure, meyer rod, slot die, flood coating, dipping, spraying, roller coating or spin coating among others and is then flashed off at room temperature and/or elevated temperature (preferably at from 20 to 200° C.). The surface of the second layer can be pretreated by cleaning or activation.

Preferably, in the second step, curing of the first layer takes place by means of UV light, there being used as the UV light source preferably a mercury vapour lamp, doped with iron, or a pure mercury vapour lamp, or one doped with gallium. Irradiation is thus carried out with light having a wavelength of 254 nm. However, curing can also take place using other UV sources such as UV LED's arrays.

By means of the dose of at least 3 J/cm², curing of the entire layer of the coating composition and incorporation of the UV absorber into the polymer matrix that forms are achieved.

The coating is applied onto a polymeric substrate typically used for coatings, such as, for example, polycarbonate, polyetheylene terepthalate, poly methyl methacrylate, poly vinylchloride, cellulose acetate. The substrates may be specialized to serve a certain purpose such as biodegradation, flame retardance, and/or hydrolysis resistance.

Polymeric substrates may have metallization and other vapor deposited or plasma deposited layers of inorganic materials.

The invention also includes a multilayer structure comprising the substrate A and a protective layer B produced by curing the composition according to the invention. Further layers are optionally possible, either on the cured composition or on the substrate before the composition according to the invention is applied. Further layers with a layer sequence B-A-B are likewise possible. The layers B can be identical or different according to the described composition.

The multilayer products according to the invention, or the thermoplastic polymers used for the production, can comprise organic dyes, inorganic dyeing pigments, fluorescent dyes and, particularly preferably, optical brighteners.

The present invention relates further to a coated substrate obtainable by a method according to the invention. The reaction of the isocyanate acrylate and the hydroxyl or amine containing UV absorber or HALS is typically 0 to 10% within the stoichiometric reaction of isocyanate groups to hydroxyl or amine moieties. Typically, the isocyanate is supplied at 0 to 5% above the stoichiometric equivalent over the hydroxyl content.

### EXAMPLES

The invention will be described further by means of the following examples, but without being limited thereto.

### Example 1

28.5 g of Tinuvin 405 (BASF) were dissolved in 154.6 g of ethyl acetate. 6.89 g of 2-isocyanatoethyl acrylate and 0.19 g of 10% w/w solution of dioctyl tin dilaurate in toluene were added to the solution. The mixture was stirred at 60° C. for 12 hours in a closed container. The residual NCO content was <0.1%, the solids content was 18.6 wt. %. The equivalent content of UV absorber was 15%.

### Example 2

28.5 g of Tinuvin 405 (BASF) were dissolved in 120.5 g of ethyl acetate. 41 g of Ebecryl 4250 (allnex) and 0.19 g of 10% w/w solution of dioctyl tin dilaurate in toluene were added to the solution. The mixture was stirred at 60° C. for 12 hours in a closed container. The residual NCO content was <0.1%, the solids content was 18.6 wt. %. The equivalent content of UV absorber was 15%.

### Example 3

28.5 g of Tinuvin 152 (BASF) were dissolved in 156.2 g of ethyl acetate. 5.3 g of 2-isocyanatoethyl acrylate and 0.19 g of 10% w/w solution of dioctyl tin dilaurate in toluene were added to the solution. The mixture was stirred at 60° C. for 12 hours in a closed container. The residual NCO content was <0.1%, the solids content was 18.6 wt. %. The equivalent content of HALS was 15%

### Example 4

For the comparison the production of a scratch-resistant coating with and without the urethane acrylate functionalized UV absorber the follow formula were analyzed the formula and results are presented in the table below:

| | Formula with | Formula with |
|---|---|---|
| | Functionalized | Standard |
| Ingredient | Absorber | Absorber |
| 1,6-Hexanediol diacrylate (HDDA) | 5.02 | 5.02 |
| Nano silica | 5.02 | 5.02 |
| Laromer 9103 | 3.50 | 3.50 |
| BYK 371 | 1.00 | 1.00 |
| Tinuvin 123 | 0.39 | 0.39 |
| Dipentaerythritol pentaacrylate | 14.21 | 14.21 |
| MEK | 49.99 | 62.49 |
| Triphenyl phosphine | 1.55 | 1.55 |
| Example 1 Acylated UV Absorber at | 16.66 | — |
| 18.6% solids | | |
| Tinuvin 405 | | 4.16 |
| UV Initiator | 2.66 | 2.66 |
| Property | | |
| UV Transmission (325 nm), % | 45 | 42 |
| Taber abrasion delta haze 7.29 9.16 | 7.3 | 9.16 |

The liquid coating formulations from the two formulas in Example 4 were applied to the Melinex 454 (Dupont) films using the Mayer rod #8 at 20 feet/min. The coatings were dried at 110° C. for 30 seconds and cured with a mercury radiator (power 80 W/cm lamp length) with a dose of about 3000 mJ/cm².

The extinction of the coating according to the invention after application to a PET film and subsequent curing was determined at 365 nm by means of a Linshang LS160 transmission meter. The scratch resistance was determined by Taber delta haze according to ASTM D1044.

The values show that, by the introduction according to the invention of the UV absorbers, high absorbing power in the UV range can be produced. At the same time the scratch resistance of the coating is improved as compared to the coating with the unmodified UV absorber. All other properties are unchanged.

The values show that the UV absorbers according to the invention, when introduced into a scratch-resistant coating, permit only a minimal change in the colour of the substrate by weathering. Unprotected substrates yellow to a considerable degree.

From the foregoing description, one ordinarily skilled in the art can easily ascertain the essential characteristics of this invention and, without departing from the spirit and scope thereof, can make various changes and modifications to the invention to adapt it to various usages and conditions.

What is claimed is:

1. A UV absorber-comprising urethane acrylate of formula (I): 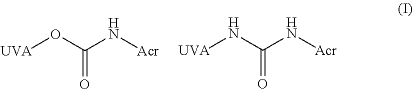
wherein
UVA is a UV absorber molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate.

2. The UV absorber-comprising urethane acrylate of claim 1, wherein the UV absorber-comprising urethane acrylate exhibits a UV absorption maximum between 300 and 360 nanometers.

3. A HALS-comprising urethane acrylate of formula (I): 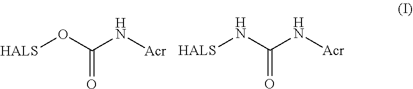
wherein
HALS is a HALS molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate.

4. A process for preparation of a UV absorber-comprising urethane acrylate, comprising the steps of:
providing a UV-absorber compound containing hydroxyl group capable of reacting with isocyanate;
reacting the UV-absorber compound containing hydroxyl with a dual-functional monomer containing acrylate and isocyanate groups according to the following reaction scheme: 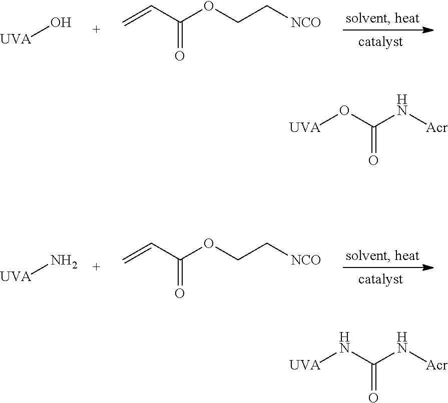
wherein,
UVA is the UV-absorber compound, and Acr is an acrylate or methacrylate group, and
wherein,
the reaction is carried out in a solvent and a catalyst is employed to accelerate the reaction.

5. The process of claim 4, wherein the dual function monomer containing acrylate and isocyanate groups has a structure according to the following formula:

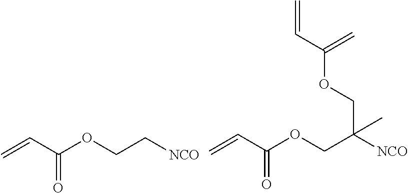

6. A process for preparation of a HALS-comprising urethane acrylate, comprising the steps:
providing a HALS compound containing hydroxyl group capable of reacting with isocyanate;
reacting the HALS containing hydroxyl with a dual-functional monomer containing acrylate and isocyanate groups according to the following reaction scheme: 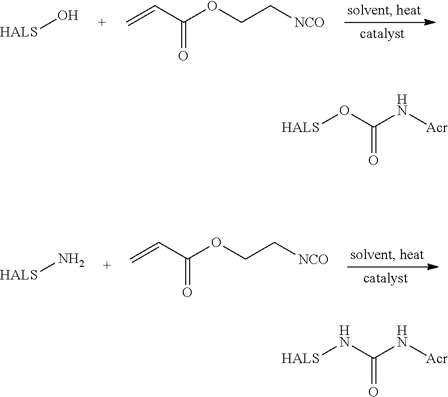
wherein,
HALS is the HALS compound, and Acr is an acrylate or methacrylate group, and
wherein,
the reaction is carried out in a solvent and a catalyst is employed to accelerate the reaction.

7. The process of claim 6, wherein the dual function monomer containing acrylate and isocyanate groups has a structure according to the following formula:

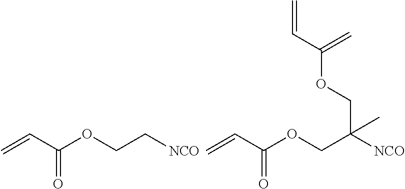

8. A coating composition comprising a UV absorber-comprising urethane acrylate according to formula (I) or a HALS-comprising urethane acrylate according to formula (II): 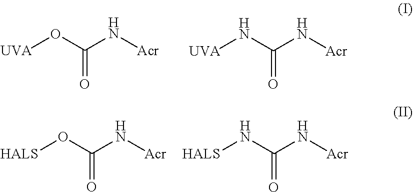
wherein
UVA is a UV absorber molecule that has a hydroxyl or an amine group reactive with isocyanate, HALS is a HALS molecule that has a hydroxyl or an amine group reactive with isocyanate, and Acr is an acrylate or methacrylate group attached through a linker selected from the group 2-isocyanatoethyl acrylate, 2-isocyanatoethyl methacrylate, and 1,1-Bis(acryloxymethyl)ethyl isocyanate.

9. The coating composition of claim 8, wherein the coating composition includes the UV absorber-comprising urethane acrylate according to formula (I) in an amount of 0 to 30 parts by weight.

10. The coating composition of claim 9, further comprising:
0 to 70 parts by weight of C₂-C₁₂-diol diacrylate or C₂-C₁₂-diol dimethacrylate, wherein C₂-C₁₂ represents a linear alkylene radical which can be substituted by a methyl group or can be interrupted by one or more oxygen atom(s) and substituted by one or more methyl group(s);
0 to 95 parts by weight of an alkoxylated mono-, di-, tri-, tetra-, penta- or hexa-acrylate or alkoxylated mono-, di-, tri-, tetra-, penta- or hexa-methacrylate;
0 to 95 parts by weight of at least one monomer selected from the group including pentaerythritol triacrylate, pentaerythritol tetraacrylate, dipentaerythritol tetraacrylate, dipentaerythritol pentaacrylate, dipentaerythritol hexaacrylate, pentaerythritol trimethacrylate, pentaerythritol tetramethacrylate, dipentaerythritol tetramethacrylate, dipentaerythritol pentamethacrylate, dipentaerythritol hexamethacrylate, and reaction products thereof with aliphatic or aromatic diisocyanates, and
0 to 95 parts by weight of at least one further mono-, di- or tri-acrylate or mono-, di- or tri-methacrylate.

11. The coating composition of claim 10, further comprising 0.1 to 10 parts by weight of at least one photoinitiator.

12. The coating composition of claim 11, wherein the coating composition is cured by irradiation with UV light.

13. The coating composition of claim 10, wherein the coating composition comprises 20 to 80 parts by weight of the at least one monomer.

14. The coating composition of claim 10, wherein the coating composition comprises 0 to 60 parts by weight of the alkoxylated mono-, di-, tri-, tetra-, penta- or hexa-acrylate or alkoxylated mono-, di-, tri-, tetra-, penta- or hexa-methacrylate

15. The coating composition of claim 8, wherein the coating composition includes the HALS-comprising urethane acrylate according to formula (II) in an amount of 0 to 25 parts by weight.

16. The coating composition of claim 15, further comprising:
0 to 70 parts by weight of C₂-C₁₂-diol diacrylate or C₂-C₁₂-diol dimethacrylate, wherein C₂-C₁₂ represents a linear alkylene radical which can be substituted by a methyl group or can be interrupted by one or more oxygen atom(s) and substituted by one or more methyl group(s);
0 to 95 parts by weight of an alkoxylated mono-, di-, tri-, tetra-, penta- or hexa-acrylate or alkoxylated mono-, di-, tri-, tetra-, penta- or hexa-methacrylate;
0 to 95 parts by weight of at least one monomer selected from the group including pentaerythritol triacrylate, pentaerythritol tetraacrylate, dipentaerythritol tetraacrylate, dipentaerythritol pentaacrylate, dipentaerythritol hexaacrylate, pentaerythritol trimethacrylate, pentaerythritol tetramethacrylate, dipentaerythritol tetramethacrylate, dipentaerythritol pentamethacrylate, dipentaerythritol hexamethacrylate, and reaction products thereof with aliphatic or aromatic diisocyanates, and
0 to 95 parts by weight of at least one further mono-, di- or tri-acrylate or mono-, di- or tri-methacrylate.

17. The coating composition of claim 16, further comprising 0.1 to 10 parts by weight of at least one photoinitiator.

18. The coating composition of claim 17, wherein the coating composition is cured by irradiation with UV light.

19. The coating composition of claim 16, wherein the coating composition comprises 20 to 80 parts by weight of the at least one monomer.

20. The coating composition of claim 16, wherein the coating composition comprises 0 to 60 parts by weight of the alkoxylated mono-, di-, tri-, tetra-, penta- or hexa-acrylate or alkoxylated mono-, di-, tri-, tetra-, penta- or hexa-methacrylate
